(19) United States
(12) Patent Application Publication
(10) Pub. No.: US 2010/0163103 A1
(43) Pub. Date: Jul. 1, 2010

(54) Organic Thin-Film Solar Cell Using Fullerene Derivative for Electron Acceptor and Method of Manufacturing the Same

(75) Inventors: Lee-Yih Wang, Taipei City (TW); Chin-Wei Liang, Taipei City (TW); Jui-Chi Lin, Tu-Chen City (TW); Wen-Hsien Ho, Tu-Chen City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: TAIWAN TEXTILE RESEARCH INSTITUTE, Tu-Chen City (TW)

(21) Appl. No.: 12/509,582

(22) Filed: Jul. 27, 2009

(30) Foreign Application Priority Data
Dec. 31, 2008 (TW) 97151710

(51) Int. Cl. H01L 31/042; H01L 51/48; C07C 69/616

(52) U.S. Cl. 136/256; 438/82; 560/80; 257/E51.039; 977/735

(57) ABSTRACT

A fullerene derivative for electron acceptor is disclosed. Introducing a benzylalkyl group into the fullerene derivative can increase the affinity of the fullerene derivative with electron donors, and introducing an alkyl group into the fullerene derivative can increase the solubility of the fullerene derivative with an organic solvent. In addition, an organic thin-film solar cell and a method of manufacturing the same are further disclosed. An annealing process can be employed to improve the crystallization and to reduce the phase separation state of a photoactive layer that is formed by the fullerene derivative and the electron acceptor. Thereby, the fullerene derivative is facilitated to enhance the solar energy to electricity conversion efficiency of the resultant organic thin-film solar cell.

### RELATED APPLICATIONS

This application claims priority to Taiwan Application Serial Number 97151710, filed Dec. 31, 2008, which is herein incorporated by reference.

### FIELD OF THE INVENTION

This invention relates generally to a fullerene derivative and a method of manufacturing the same, and more particularly, to a fullerene derivative for electron acceptor and its application of an organic thin-film solar cell manufactured by using the same.

### BACKGROUND OF THE INVENTION

Since people raise the environmental awareness and other petroleum-related energies are going to exhaust, it is indeed necessary to develop a new and safe energy. A new energy must satisfy at least two requirements for worth developing, one of which is rich in reserves and difficulty exhausted, and the other of which is safe, clean, and friendly to human beings and nature environment. Regenerative energy, for example, solar energy, wind power, water power and so on, can satisfies the above two requirements. Besides, Taiwan lacks natural energy resources, and more than 90% of the required energy must be imported from other countries. However, Taiwan has enough sunlight and more insolation due to its location in the subtropical zone. Thus, it is advantageous to research and develop the solar energy in Taiwan, and the electric power converted by the solar energy is beneficial to save energy and environmental protection.

Utilizing solar cells (also called photovoltaic devices) is a direct way to convert the solar energy to energy. Nowadays, silicon (Si) semiconductor materials are utilized to produce most of commercialized solar cells. Si-based semiconductor materials are divided to single-crystal, poly-silicon, amorphous Si and so on according to the Si crystal states. The solar cells fabricated by using single-crystal Si have higher and stable energy conversion efficiency but cost expensively. The solar cells fabricated by using amorphous Si have lower energy conversion efficiency and shorter lifespan. Therefore, the dye-sensitized solar cell (DSSC) fabricated by organic materials such as polymers are more important to the academic and industrial circles.

Organic polymer-semiconductor film (in general, approximately 100 nm thickness) is directly employed to a photosensitive and optoelectrical conversion material in the organic thin-film solar cell, and thus the organic polymer-semiconductor film is one of the key technologies for developing the organic thin-film solar cell. Under light irradiation, the semiconductor layer absorbs photons to generate electron-hole pairs (excitons) and photoinduced charge transfer, resulting in separation of electrons and holes. Next, the anode electrode collects the holes through the hole-transferring layer, so as to generate photocurrent.

However, the organic polymer has an inherent disadvantage in higher bonding energy of electron-hole pairs, and it is very hard to generate free electron-hole pairs at room temperature. In order to separate electron-hole pairs more effectively, Dr. C. W. Tang successfully separated electron-hole pairs by employing the concept of P-N junction in 1986, and the optoelectrical conversion of the organic photovoltaic device is elevated to approximately 1%. In 1992, the team leded by A. J. Heeger and F. Wudi further found that, the electron-hole pairs can be effectively separated by using the mixture of electron donor (such as p-type conjugated polymer) and electron acceptor (n-type organic material), so as to enhance optoelectrical conversion efficiency. Nowadays, the popular n-type organic material is fullerene derivative, for example, [6,6]-phenyl C₆₁-butyric acid methyl ester (PCBM).

The device efficiency of the organic thin-film solar cell can be improved by new structure (for example, the solar cell with tandem structure) or processing conditions, in addition to develop new p-type conjugated polymer and n-type organic material. However, since PCBM is the mainly n-type organic material but other alternative materials are less, it is disadvantageous to Taiwan to independently develop the organic thin-film solar cell.

Hence, it is necessary to provide an n-type organic material as electron acceptor, so as to provide more choices about alternative materials applied in the organic thin-film solar cell.

### SUMMARY OF THE INVENTION

Accordingly, it is an aspect of the present invention to provide a fullerene derivative for electron acceptor. The fullerene derivative introduces a benzylalkyl group for increasing its affinity of the fullerene derivative with electron donors, and it introduces an alkyl group for increasing its solubility in an organic solvent. Therefore, the fullerene derivative for electron acceptor can be availably applied to an organic thin-film solar cell.

It is another aspect of the present invention to provide an organic thin-film solar cell and a method of manufacturing the same. The organic thin-film solar cell has fullerene derivative for electron acceptor. The fullerene derivative introduces a benzylalkyl group for increasing its affinity of the fullerene derivative with electron donors, and it introduces an alkyl group for increasing its solubility in an organic solvent. Additionally, an annealing process can be employed to improve the crystallization and to reduce the phase separation state of the fullerene derivative and the electron acceptor, so as to enhance the solar energy to electricity conversion efficiency of the resultant organic thin-film solar cell.

According to the aforementioned aspect of the present invention, a fullerene derivative for electron acceptor is provided and represented by a chemical formula (I) as follows:

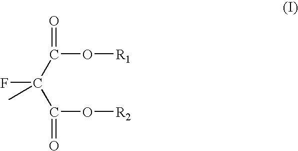

in which F may be fullerene, R₁ may be independently selected from the group consisting of straight, branched or cyclic-chained C₂₋₁₀ alkyl groups, R₂ may be a group of C₆H₅—CₙH₂ₙ—, and n of the R₂ may be 1 to 3.

According to another aforementioned aspect of the present invention, an organic thin-film solar cell is provided. In an embodiment, the organic thin-film solar cell may include a light-transmitting electrode, a hole-transferring layer, a photoactive layer and a metal electrode, each of which is sequentially disposed. In an embodiment of the present invention, electron acceptor and electron donor, and the electron acceptor is represented by a chemical formula (I) as follows:

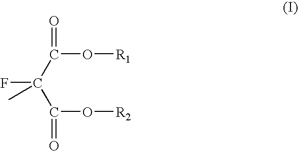

in which F may be fullerene, R₁ may be independently selected from the group consisting of straight, branched or cyclic-chained C₂₋₁₀ alkyl groups, R₂ may be a group of C₆H₅—CₙH₂ₙ—, and n of the R₂ may be 1 to 3.

In an embodiment of the present invention, the electron donor may be a conjugated polymer, for example, poly(3-hexylthiophene) (P3HT).

In an embodiment of the present invention, a weight ratio of the electron donor to the fullerene derivative may be 1:0.2 to 1:5, for example.

In an embodiment of the present invention, the hole-transferring layer may include poly(3,4-ethylenedioxy-thiophene) (PEDOT): poly(styrene sulfonate) (PSS).

According to the aforementioned aspect of the present invention, a method of manufacturing organic thin-film solar cell is further provided. In an embodiment, a photoactive layer may be formed on a light-transmitting electrode, in which the photoactive layer may include electron donor and electron acceptor, and the electron acceptor may be represented by a chemical formula (I) as above, in which F may be fullerene, R₁ may be independently selected from the group consisting of straight, branched or cyclic-chained C₂₋₁₀ alkyl groups, R₂ may be a group of C₆H₅—CₙH₂ₙ—, and n of the R₂ may be 1 to 3. Next, a metal electrode may be fanned on the photoactive layer.

In an embodiment of the present invention, the method of manufacturing the organic thin-film solar cell may further include to form a hole-transferring layer on the light-transmitting electrode, and the hole-transferring layer may include PEDOT: PSS.

In an embodiment of the present invention, the method of manufacturing the organic thin-film solar cell may further include to perform an annealing step after the photoactive layer is formed, in which the annealing step may be performed under 20° C. to 250° C. for 1 minute to 60 minutes, for example.

With application to the aforementioned fullerene derivative for electron acceptor and the organic thin-film solar cell manufactured by using the same in the present invention, the fullerene derivative introduces a benzylalkyl group for increasing its affinity of the fullerene derivative with electron donors, and it introduces an alkyl group for increasing its solubility in an organic solvent. Additionally, an annealing process can be employed to improve the crystallization and to reduce the phase separation state of the fullerene derivative and the electron acceptor, so as to improve the state of the photoactive layer of the organic thin-film solar cell, as well as enhancing the solar energy to electricity conversion efficiency of the resultant organic thin-film solar cell.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a ¹H-NMR spectrum of the first intermediate product dissolved in deuterated chloroform (CDCl₃) according to an embodiment of the present invention.

FIG. 2 is a ¹H-NMR spectrum of the second intermediate product dissolved in CDCl₃ according to an embodiment of the present invention.

FIG. 3 is a ¹H-NMR spectrum of the BBMDC dissolved in CDCl₃ according to an embodiment of the present invention.

FIG. 4 is a ¹³C-NMR spectrum of the BBMDC dissolved in CDCl₃ according to an embodiment of the present invention.

FIG. 5 is an UV-vis absorption spectrum of the fullerene C₆₀ dissolved in CDCl₃ according to an embodiment of the present invention.

FIG. 6 is an UV-vis absorption spectrum of the BBMDC dissolved in CDCl₃ according to an embodiment of the present invention.

FIG. 7 is a HPLC chromatogram of the BMDC according to an embodiment of the present invention.

FIGS. 8(a) to 8(e) depict top views of the organic thin-film solar cell process according to an embodiment of the present invention.

FIG. 9 is an UV-vis absorption spectrum of the photoactive layer according to an embodiment of the present invention.

FIG. 10 is an UV-vis absorption spectrum of the photoactive layer according to an embodiment of the present invention.

FIG. 11 is a photoluminescence spectrum of the photoactive layer according to the present invention.

FIG. 12 is a current-voltage curve of the organic thin-film solar cell according to an embodiment of the present invention.

FIG. 13 is an external quantum efficiency (EQE) spectrum of the organic thin-film solar cell according to an embodiment of the present invention.

### DETAILED DESCRIPTION OF THE PREFERRED EMBODIMENT

Accordingly, the present invention provides a fullerene derivative for electron acceptor and an organic thin-film solar cell manufactured by using the same in the present invention, the fullerene derivative introducing a benzylalkyl group an alkyl group therein servers as an n-type polymer for electron acceptor. The benzylalkyl group is available to increase its affinity of the fullerene derivative with electron donors, and the alkyl group is available to increase its solubility in an organic solvent. Thereby, the fullerene derivative is facilitated to enhance the solar energy to electricity conversion efficiency of the resultant organic thin-film solar cell.

Fullerene Derivative for Electron Acceptor and Method of Manufacturing the Same

In detail, the fullerene derivative for electron acceptor may be represented by a chemical formula (I) as follows:

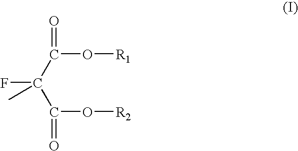

in which F may be fullerene, R₁ may be independently selected from the group consisting of straight, branched or cyclic-chained C₂₋₁₀ alkyl groups, R₂ may be a group of C₆H₅—CₙH₂—, and n of the R₂ may be 1 to 3. In another embodiment, the F may be C₆₀₋₈₄ fullerene, the R₁ may be independently selected from the group consisting of straight-chained C₄₋₁₀ alkyl groups, and the n of the R₂ may be 1. In a further embodiment, the F may be C₆₀ fullerene, the R₁ may be a butyl group, and the n of the R₂ may be 1.

In an embodiment, the fullerene derivative for electron acceptor may be synthesized as follows. First of all, 2,2-dimethyl-1,3-dioxane-4,6-dione, which is represented by a chemical formula (IV) and synthesized by reacting malonic acid and acetone, for example, may be reacted with a first alcohol, so as to obtain a first intermediate product, in which the first intermediate product may be represented by a chemical formula (II):

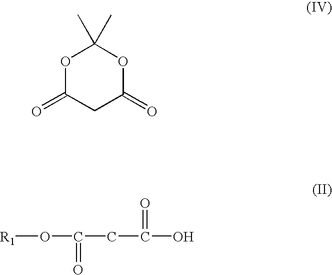

in which R₁ may be independently selected from the group consisting of straight-chained C₂₋₁₀ alkyl groups.

Next, a second alcohol may be esterified with the first intermediate product, so as to obtain a second intermediate product, in which the second intermediate product may be a malonic ester derivative, and the second intermediate product may be represented by a chemical formula (III) as follows:

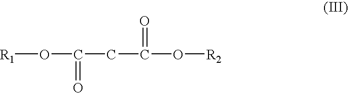

in which R₂ may be a group of C₆H₅—CₙH₂ₙ—, and n of the R₂ may be 1 to 3, for example.

Later, the second intermediate product and a fullerene may be subjected to perform a Bingel reaction, so as to obtain the fullerene derivative represented by chemical formula (I) as above.

The resulted fullerene derivative may serve as electron acceptor of an organic thin-film solar cell. The fullerene derivative can be applied to the organic thin-film solar cell and a method of manufacturing the sane exemplified as follows.

Organic Thin-Film Solar Cell and Method of Manufacturing the Same

In an embodiment, the organic thin-film solar cell may include a light-transmitting electrode, a hole-transferring layer, a photoactive layer and a metal electrode, each of which is sequentially disposed. However, as is understood by a person skilled in the art, the light-transmitting electrode, the hole-transferring layer, the photoactive layer and the metal electrode of the present invention described as above are illustrated of the present invention rather than limiting of the present invention. It is intended to cover various modifications and similar arrangements included within the spirit and scope of the appended claims. In an embodiment, the photoactive layer may include electron donor and electron acceptor, in which the electron donor may be a conjugated polymer of p-type conjugated polymer, and the electron acceptor may be the n-type fullerene derivative.

In an embodiment, the organic thin-film solar cell may be manufactured as follows. First of all, a hole-transferring layer, for example, poly(3,4-ethylenedioxy-thiophene) (PEDOT): poly(styrene sulfonate) (PSS), may be formed on a light-transmitting electrode by using spin-coating or blade-coating. The light-transmitting electrode may be a patterned indium-tin-oxide (ITO) or fluorine-doped tin oxide (FTO) circuit formed on a transparent substrate. Besides, the surface of the transparent substrate may be optionally cleaned and treated by plasma, for example.

And then, a photoactive layer may be formed on the hole-transferring layer by using spin-coating or blade-coating. The photoactive layer may include a fullerene derivative and a p-type conjugated polymer, in which the p-type conjugated polymer may be poly(3-hexylthiophene) (P3HT), for example, and the fullerene derivative is represented by chemical formula (I) as above rather than addressing the related details herein. In an embodiment, a weight ratio of the p-type conjugated polymer to the fullerene derivative may be 1:0.2 to 1:5, for example. On another embodiment, the weight ratio of the p-type conjugated polymer to the fullerene derivative may be 1:1 approximately.

Subsequently, an annealing step may be performed under 20° C. to 250° C. for 1 minute to 60 minutes, for example. In another embodiment, the annealing step may be performed under 100° C. to 150° C. for 5 minutes to 20 minutes.

It is worth mentioning that, the phase separation of the p-type conjugated polymer and the fullerene derivative blended in the photoactive layer is a key factor. The weight ratio of the p-type conjugated polymer to the fullerene derivative and the annealing condition are both facilitated to generate more p-n interface in the p-type conjugated polymer and to separate the electron-hole pairs (excitons) effectively. On one hand, the weight ratio of the p-type conjugated polymer to the fullerene derivative can change the mobility rate of the electron-hole pairs. On the other hand, the annealing condition can improve the crystallization of the obtained hole-transferring material and reduce the phase separation state of electron donor and electron acceptor, so as to enhance the mobility rate of the electron-hole pairs.

After the annealing step is finished, a metal electrode, which may be made of aluminum or calcium, is formed on the photoactive layer by using vapor evaporation method, for example.

Thereinafter, various applications of the present invention will be described in more details referring to several exemplary embodiments below, while not intended to be limiting. Thus, one skilled in the art can easily ascertain the essential characteristics of the present invention and, without departing from the spirit and scope thereof, can make various changes and modifications of the invention to adapt it to various usages and conditions.

### Example 1

### Synthesis of Fullerene Derivative

EXAMPLE 1 is related to synthesize the first and second intermediate products for preparing a fullerene derivative.

1. Synthesis of the First Intermediate Product

Firstly, 2,2-dimethyl-1,3-dioxane-4,6-dione, which is represented by a chemical formula (IV) (0.5649 g, 3.92 mmol), may be reacted with n-butyl alcohol (0.3712 g, 5 mmol) in a round-bottomed bottle under 110° C. to 120° C. for about 3 hours. After un-reactive n-butyl alcohol is removed by using low-pressure distillation method, the crudely first intermediate product (yellow liquid) is remained and purified by column chromatography (eluent: dichloromethane, CH₂Cl₂), so as to obtain the first intermediate product (colorless liquid) represented by a chemical formula (VI) (0.5337 g, 3.33 mmol) with yield of about 85%.

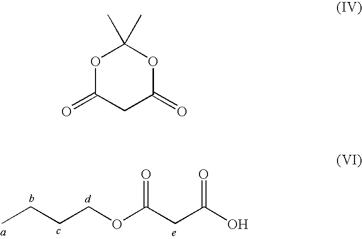

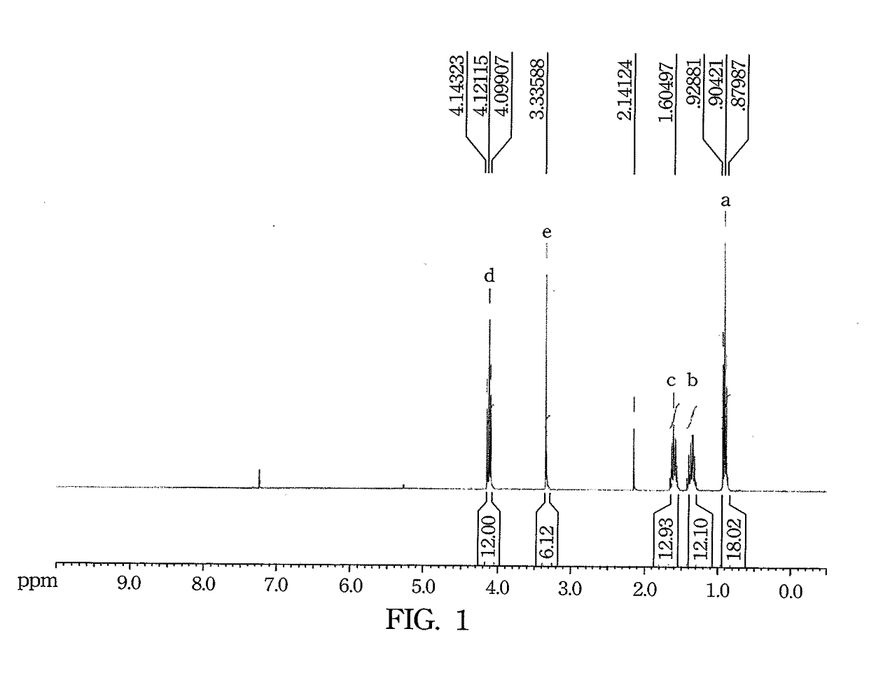

Later, the first intermediate product is further analyzed by ¹H-NMR. Reference is made to FIG. 1, which is a ¹H-NMR spectrum of the first intermediate product dissolved in deuterated chloroform (CDCl₃) according to an embodiment of the present invention. According to the result of FIG. 1, the signals of the chemical shifts occur at δ=0.90 (triplet, sharp), δ=1.40 (multiplet, broad), δ=1.60 (multiplet, broad), δ=4.12 (triplet, sharp) and δ=3.33 (singlet, sharp) corresponding to the hydrogen atoms at a to e positions of the chemical formula (VI) of the first intermediate product respectively. Also, the characteristics of integrals and splitting arising from those signals correspond to the ratio of the chemical formula (VI), which directly evidence the structure of the first intermediate product.

2. Synthesis of the Second Intermediate Product

Firstly, the first intermediate product (0.5337 g, 3.33 mmol), benzyl alcohol (0.4863 g, 4.5 mmol), 1-hydroxybenzotriazole (45 mg, 0.33 mmol, 10% w/w), anhydrous tetrahydrofuran (THF; 5 ml) and anhydrous dichloromethane (CH₂Cl₂; 20 ml) are added with N,N-dicyclohexylcarbodiimide (DCC; 0.8253 g, 4 mmol) in dichloromethane solution (5 ml) under cold-bath condition, stirred for about 1 hour, and heated to about 40° C. in oil bath for about 24 hours. Next, the precipitate of dicyclohexyl urea (DCU) is removed by using vacuum filtration method, and the remaining solvent is removed by using a rotary concentrator, for example, so as to obtain the crudely second intermediate product. The crudely second intermediate product is further purified by using column chromatography (eluent: dichloromethane, CH₂Cl₂), so as to obtain the second intermediate product (yellowish liquid) represented by a chemical formula (VII) (0.6668 g, 2.67 mmol) with yield of about 83%.

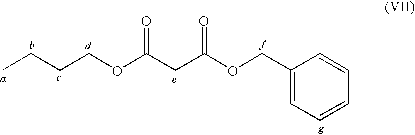

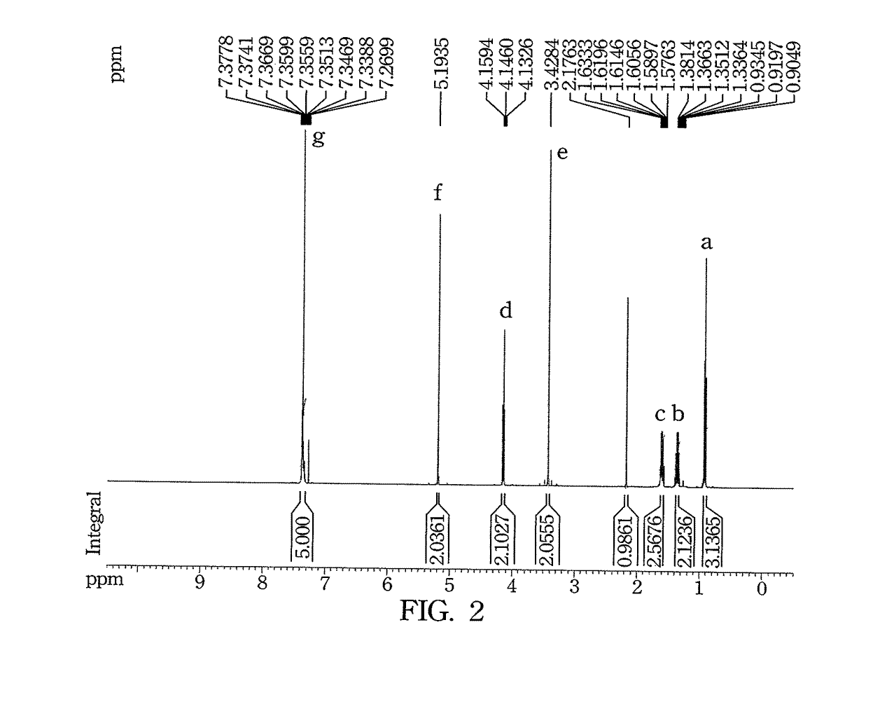

Later, the second intermediate product is further analyzed by ¹H-NMR. Reference is made to FIG. 2, which is a ¹H-NMR spectrum of the second intermediate product dissolved in CDCl₃ according to an embodiment of the present invention. According to the result of FIG. 2, the signals of the chemical shifts occur at δ=0.90 (triplet, sharp), δ=1.32 (multiplet, broad), δ=1.60 (multiplet, broad), δ=4.05 (triplet, sharp), δ=3.33 (singlet, sharp) δ=5.08 (singlet, sharp) and δ=7.26 (singlet, broad) corresponding to the hydrogen atoms at a to g positions of the chemical formula (VII) of the second intermediate product respectively. Also, the characteristics of integrals and splitting arising from those signals correspond to the ratio of the chemical formula (VII), which directly evidence the structure of the second intermediate product.

3. Synthesis of (benzylbutyl)(1,2-methanofullerene C₆₀)-61,61-dicarboxylate (BBMDC)

Firstly, the second intermediate product (87.6 mg, 0.35 mmol), fullerene C₆₀ (200 mg, 0.28 mmol), iodine (I₂; 88.8 mg, 0.35 mmol) are added with anhydrous toluene (200 ml) in a round-bottomed bottle, stirred for about 1 hour, added with 1,5-dizzabicycloundecen-5-ene (DBU; 60.8 mg, 0.4 mmol) in toluene solution (10 ml) and reacted for about 5 hours. Next, the DBU is removed by using vacuum filtration method, and the solvent is removed by using a rotary concentrator, for example, so as to obtain the crude BBMDC. The crude BBMDC is further purified by using column chromatography (eluent: dichloromethane, CH₂Cl₂), so as to obtain the BBMDC represented by a chemical formula (VIII) (94.9 mg) with yield of about 35%.

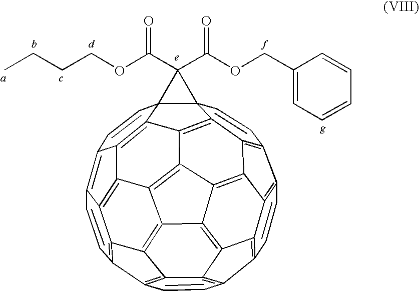

Later, the BBMDC is further analyzed by ¹H-NMR. Reference is made to FIG. 3, which is a ¹H-NMR spectrum of the BBMDC dissolved in CDCl₃ according to an embodiment of the present invention. According to the result of FIG. 3, the signals of the chemical shifts occur at δ=0.90 (triplet, sharp), δ=1.43 (multiplet, broad), δ=1.70 (multiplet, broad), δ=4.44 (triplet, sharp), δ=7.42 (multiplet, broad), and δ=5.52 (singlet, sharp) corresponding to the hydrogen atoms at a, b, c, d, f and g positions of the chemical formula (VIII) of the BBMDC respectively. Also, the characteristics of integrals and splitting arising from those signals correspond to the ratio of the chemical formula (VIII), which directly evidence the structure of the BBMDC. Furthermore, the most important thing is that, the Bingel reaction is indeed carried out and evidenced partially by the featured peak at δ=3.33 (i.e. hydrogen atom at e position) disappeared by this reaction according to this NMR spectrum.

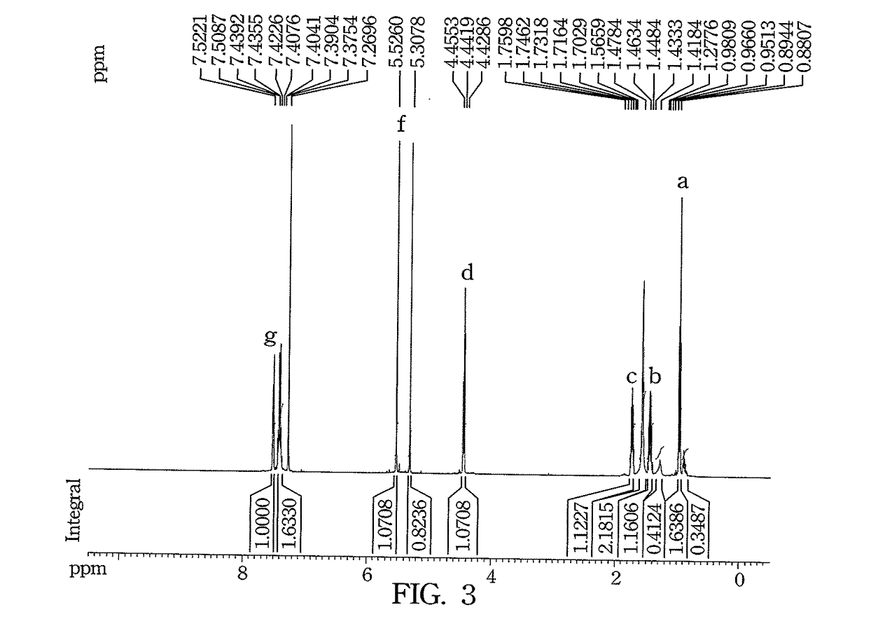

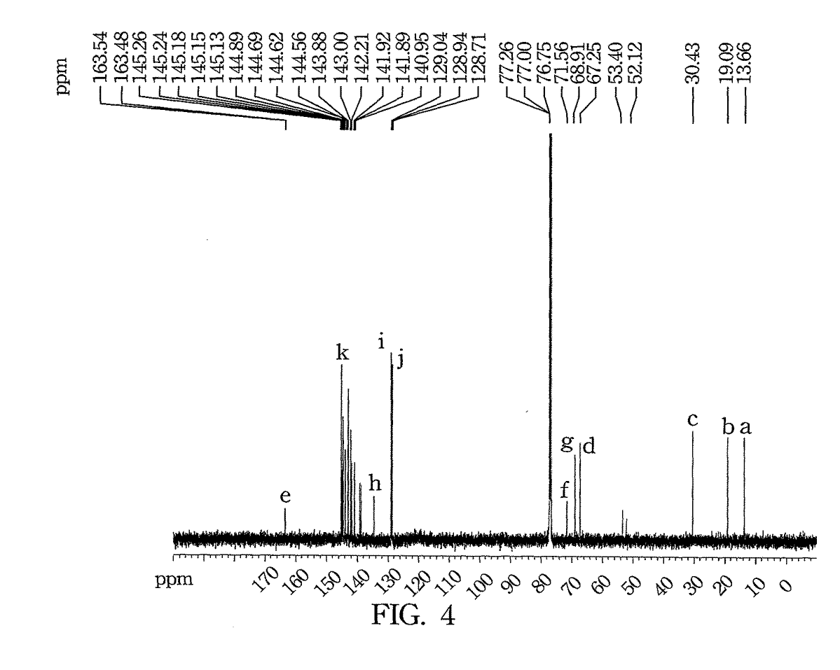

Besides, the BBMDC is further analyzed by ¹³C-NMR. Reference is made to FIG. 4, which is a ¹³C-NMR spectrum of the BBMDC dissolved in CDCl₃ according to an embodiment of the present invention. According to the result of FIG. 4, as the chemical formula (IX) of the BBMDC, the signals of the chemical shifts occur at δ=13.65, δ=19.09, δ=30.43, δ=67.25, δ=68.91, δ=163.54, δ=71.55, δ=140.95, δ=129.04, δ=128.71, and δ=144.62 respectively corresponding to the carbon atoms at a, b, c, d, g, e, f, h, i, j and k positions of the chemical formula (IX) of the BBMDC. It is found that, among the shifting peaks of various carbon atoms of the ¹³C-NMR spectrum of the BBMDC, the featured peak at δ=71.55 (i.e. carbon atom at f position) is referred to the three-membered ring, which has sp3 hybrid orbital and is reacted by the second intermediate product with the fullerene C₆₀, thereby evidencing that the BBMDC is indeed synthesized.

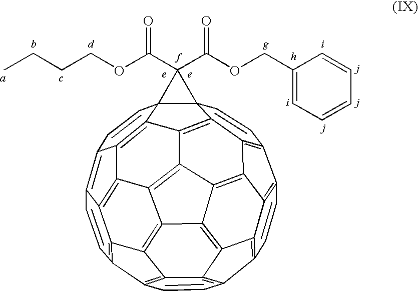

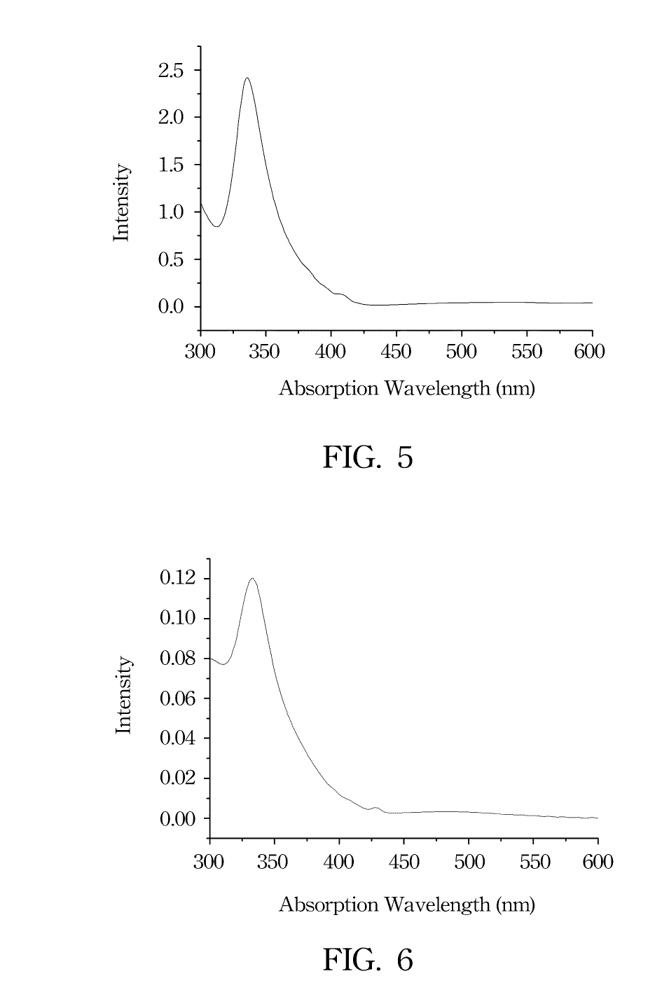

In addition, the BBMDC is further analyzed by ultraviolet-visible (UV-vis) absorption spectra. Reference is made to FIGS. 5 and 6. FIG. 5 is an UV-vis absorption spectrum of the fullerene C₆₀ dissolved in CDCl₃, and FIG. 6 is an UV-vis absorption spectrum of the BBMDC dissolved in CDCl₃ according to an embodiment of the present invention, in which the vertical axes of FIGS. 5 and 6 are referred to an intensity, and the horizontal axes of FIGS. 5 and 6 are referred to absorption wavelength (nm). According to the result of FIGS. 5 and 6, the fullerene C₆₀ has the featured absorption peaks at λ=340 nm and λ=410 nm in FIG. 5, but the featured peak at λ=410 nm is red-shifted to λ=430 nm in FIG. 6. The observed red shift may be explained by decreased numbers of the broken conjugated double bonds of the fullerene C₆₀.

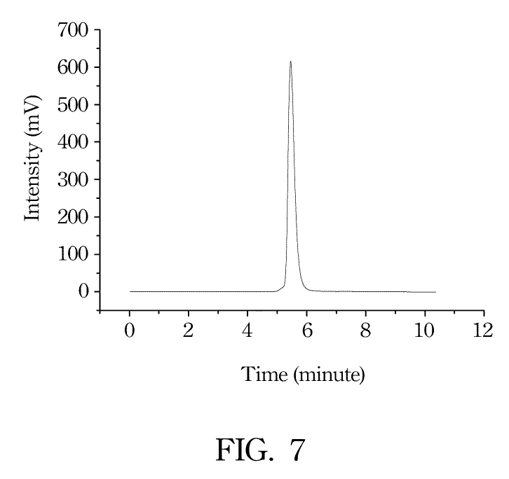

Furthermore, the BBMDC may be analyzed by high performance liquid chromatography (HPLC). Reference is made to FIG. 7, which is a HPLC chromatogram of the BMDC according to an embodiment of the present invention, in which the vertical axis is referred to measured signal intensity (mV), and the horizontal axis is referred to time (minute). The HPLC operation condition in FIG. 7 further includes that the BBMDC is subjected to pass through the HPLC column in a flow rate of 0.5 mL per second, and the absorption variation of the BBMDC at 340 nm is detected in a certain time interval. The synthesized BBMDC is a single substance evidenced by a sharply single peak in FIG. 7, and its purity was more than 99.9%.

### Example 2

### Preparation of Organic Thin-Film Solar Cell

EXAMPLE 2 is related to prepare an organic thin-film solar cell including a light-transmitting electrode, a photoactive layer and a metal electrode, in which the photoactive layer may include the BBMDC of EXAMPLE 1 and P3HT.

1. Cleaning, Patterning and Surface Treatment of Light-Transmitting Electrode

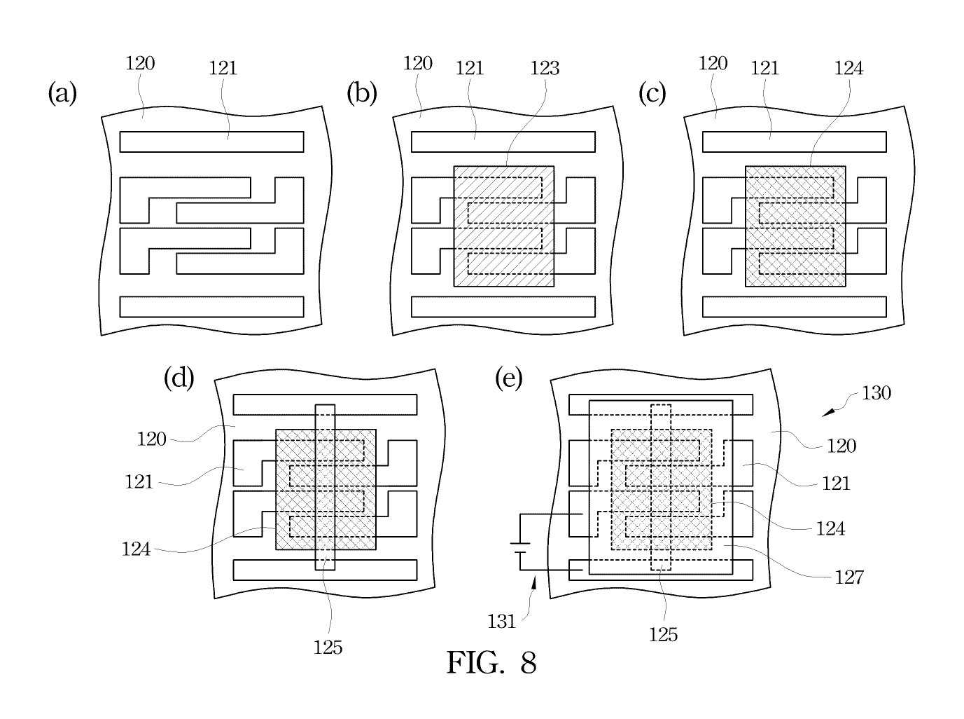

First, in this example, the glass substrate coated with ITO transparent conductive film is cut into a desired size for forming a conductive glass 120 shown in FIG. 8(a). And then, the fingerprints on the conductive glass with the desired size are cleaned with a glass detergent and ultrasonicated in the glass detergent (for example, Triton X 100) for about 10 minutes. Later, the conductive glass is rinsed and ultrasonicated in deionized water, ultrasonicated in acetone for about 10 minutes, and ultrasonicated in isopropanol for about 10 minutes. After the conductive glass is dried by low-pressure nitrogen gas, a multimeter detects the conductive side of the conductive glass, and the other side opposing the conductive side is marked.

Next, the light-transmitting electrode is patterned. In detail, the positive photoresist (for example, AZ1500) is firstly coated on the conductive glass by spin-coating method and the first spinning velocity is 1000 rpm for 5 seconds, the second one is 3000 rpm for 40 seconds, for example. Following, the conductive glass may be placed on the hot plate and baked at 100° C. for 10 minutes, for example, so as to remove the solvent in the positive photoresist and to increase adhesion of the positive photoresist. Later, the positive photoresist on the conductive glass is exposed to a desired patterned mask with UV exposure machine for 60 seconds (exposure intensity: approximately 4.5 mW/cm²). After the exposure process is finished, the conductive glass is immersed into a developer solution (for example AZ400K) for 10 seconds, taken out and vigorously washed in water, so as to prevent the photoresist from remaining. And then, the conductive glass may be placed on the hot plate again and baked at 140° C. for 10 minutes, for example. Afterward, the conductive glass is etched by using 37 wt. % of hydrochloric acid (HCl) for 180 seconds so as to remove undesired ITO patterns. Subsequently, the remaining positive photoresist on the conductive glass is dissolved in acetone, so as to form a conductive electrode 121 shown in FIG. 8(a). The etching and shortcut states with respect to the conductive electrode 121 are further verified by an optically microscopical observation and a multimeter measurement, respectively.

And then, the aforementioned conductive glass is subjected to surface treatment. Firstly, the etched conductive glass is cut into a size of 2 square centimeters (cm²) and ultrasonicated in the glass detergent (for example, Triton X 100) for about 10 minutes. Later, the conductive glass is ultrasonicated twice in the deionized water, ultrasonicated in acetone for about 10 minutes, and ultrasonicated in isopropanol for about 10 minutes. After the conductive glass is dried by low-pressure nitrogen gas, a multimeter detects the conductive side of the conductive glass, and the other side opposing the conductive side is marked.

Subsequently, the aforementioned conductive glass is subjected to surface cleaning, for removing undesired organic pollutant and increasing the hydrophilicity on the surface of the conductive glass. This process facilitates to coat the hole-transferring layer (for example, PEDOT: PSS). In an embodiment, the surface cleaning process was conducted by using oxygen plasma under the following conditions, for example, a plasma power of approximately 50 W, a plasma ignition pressure of 200 mtorr to 300 mtorr, and a cleaning period of approximately 3 minutes.

2. Formation of Hole-Transferring Layer

At first, the suitable material of the hole-transferring layer may be, for example, PEDOT: PSS solution (BAYTRON® P VP AI 4083, H. C. Starck Co.). The PEDOT: PSS solution is ordinarily stored at low temperature but thawed before use. Next, the PEDOT: PSS solution is coated on the surface-treated conductive glass by using spin-coating or blade-coating method, for example, a rotary speed of 3500 rpm for 40 seconds in the spin-coating method. After coating the PEDOT: PSS layer, the redundant PEDOT: PSS layer can be removed by water, and the hole-transferring layer 123 is subsequently remained as shown in FIG. 8(b). Later, the conductive glass coated with the PEDOT: PSS layer is placed on the hot plate and baked at 140° C. for 20 minutes, for example, so as to remove the moisture therein and obtain the hole-transferring layer of approximately 30 nm thickness.

3. Formation of Photoactive Layer

Following, P3HT (15 mg) blended with BBMDC(X mg) of EXAMPLE 1 in various ratios are dissolved in 1.0 mL of chlorobenzene and stirred at 40° C. for 12 hours, and then the P3HT/BBMDC solution is coated on the hole-transferring layer (PEDOT: PSS) by using spin-coating or blade-coating method, for example, under a rotary speed of 1000 rpm for 30 seconds in the spin-coating method. After coating the P3HT/BBMDC layers, the redundant P3HT/BBMDC layers can be removed by acetone, and the remained P3HT/BBMDC layers are as the photoactive layer 124 shown in FIG. 8(c).

4. Annealing Process

The aforementioned conductive glass having hole-transferring and photoactive layers thereon is subjected to an annealing process at various temperatures for approximately 10 minutes, for example, room temperature, 100° C., 150° C. or 200° C.

5. Formation of Metal Electrode

Subsequently, the annealed conductive glass is subjected to a vapor evaporation process in a vacuum evaporation apparatus, which uses an aluminum target and a mask (for example, stainless steel mask) under a pressure of approximately 4×10⁻⁶ torr, so as to form an aluminum metal electrode 124 of approximately 100 nm thickness shown in FIG. 8(d). The resulted electrode may include hole-transferring layer 123, the photoactive layer 124 and the metal electrode 125 sequentially disposed on the conductive glass 120, and the hole-transferring layer 123 and the photoactive layer 124 are located in the desired light-absorbing region (not shown). After the evaporation process is finished, the resulted electrode is cooled down to room temperature, and introducing highly pure nitrogen gas breaks the vacuum state. A cover glass 127 shown in FIG. 8(e) is put over and adhered with the resulted electrode by using UV adhesive that is hardened by UV irradiation, thereby finishing the assembly of the organic thin-film solar cell 130 shown in FIG. 8(e), in which an external power 131 can be subsequently employed to evaluate the optoelectrical characteristics of the resulted organic thin-film solar cell 130.

### Example 3

### Evaluation of Optoelectrical Characteristics of Organic Thin-Film Solar Cell

EXAMPLE 3 is related to evaluate optoelectrical characteristics of the organic thin-film solar cell of EXAMPLE 2, for example, short circuit current (Isc), open circuit voltage (Voc), fill factor (FF) and solar energy to electricity conversion efficiency (η). In this example, a 450 W xenon (Xe) short arc lamp (Lot-Oriel Ltd.) serves as a light source of the system for evaluating the organic thin-film solar cell performance, a filter serves to modify the spectral output of the Xe short arc lamp to match solar conditions for providing a simulated solar radiation, and a photodetector (Optical Power meter, Solar Light Company, Inc. PMA-2141) serves to adjust the simulated solar radiation to 100 W/cm² of light intensity. After the light source is stable, the organic thin-film solar cell of EXAMPLE 2 is irradiated under the beam from the adjusted light source and electrically connected to positive and negative terminals of a power supply that provides a positive voltage controlled by a sourcemeter. The output current of the organic thin-film solar cell is measured to obtain a current-voltage (I-V) curve and optoelectrical characteristics, for example, short circuit current (Isc), open circuit voltage (Voc), fill factor (FF) and solar energy to electricity conversion efficiency (η).

The “short circuit current (Isc)” herein is referred to a working current of a solar cell under the short circuit condition, and also referred to a “short circuit light current”, which is equal to an absolute quantity of photons converting to electron-hole pairs, while the output voltage of the solar cell is zero. Typically, the higher short circuit current of the solar cell is better.

The “open circuit voltage (V_{oc})” herein is the voltage across the positive and negative terminals under open-circuit conditions, and the current is zero, which corresponds to a load resistance of infinity. Typically, the higher open circuit voltage of the solar cell is better.

The “short circuit current (I_{sc})” herein is the current produced when the positive and negative terminals of the cell are short-circuited, and the voltage between the terminals is zero, which corresponds to a load resistance of zero. Typically, the higher open circuit voltage of the solar cell is better.

The “fill factor (FF)” herein is referred to a ratio of a maximum output power (Pₘₐₓ=(I×V)ₘₐₓ) of a solar cell circuit, with respect to a output power (the multiplied product of V_{oc} and I_{sc}) of a solar cell, as the following formula (I). Typically, the expected value of the fill factor of a solar cell is 1, but the actual one is less than 1.

$$FF = \frac{{P}_{max}}{{I}_{sc} \times {V}_{oc}} = \frac{{( I \times V )}_{max}}{{I}_{sc} \times {V}_{oc}} \qquad ( I )$$

The “solar energy to electricity conversion efficiency (η)” herein is referred to a percentage of a maximum output power (Pₘₐₓ) of a light receiving unit area of a solar cell with respect to an energy density of the emitted sunlight (P_{light}), and it is obtained by the following formula (II). The higher solar energy to electricity conversion efficiency is better:

$$η ( \% ) = \frac{{( I \times V )}_{max}}{{P}_{light}} \times 100 \% \qquad ( II )$$

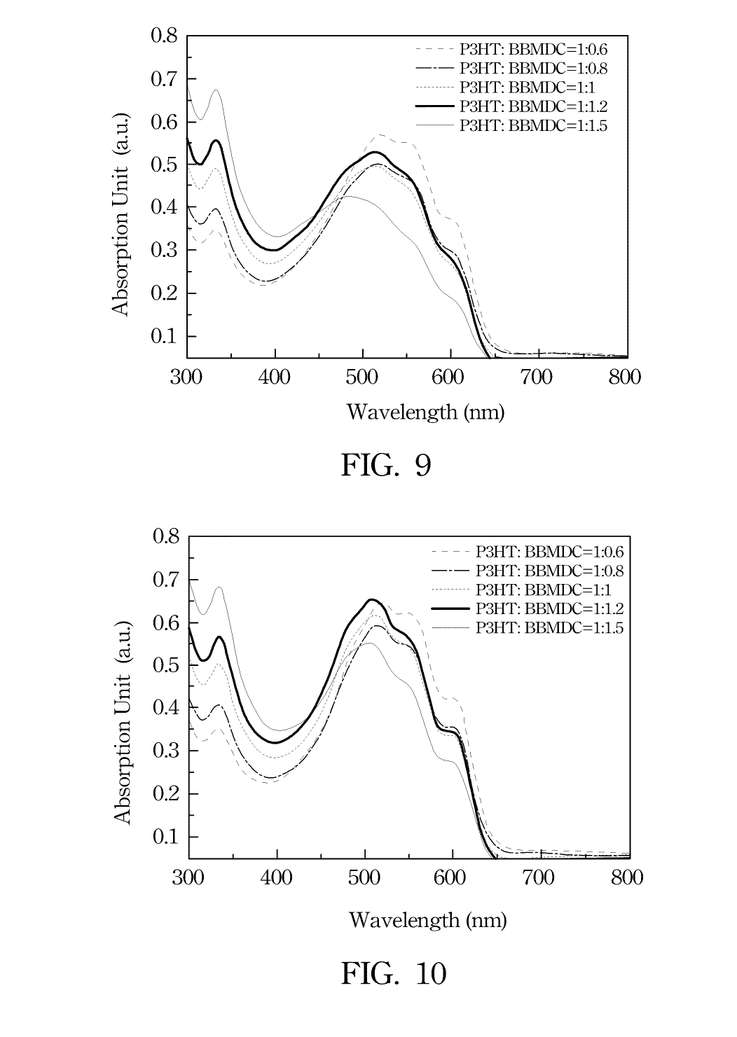

Reference is made to FIG. 9, which is an UV-vis absorption spectrum of the photoactive layer according to an embodiment of the present invention, and the photoactive layer is obtained by blending P3HT and BBMDC of EXAMPLE 2 in a weight ratio of 1:0.6, 1:0.8, 1:1, 1:1.2, or 1:1.5 without annealing treatment. The vertical axis of FIG. 9 is referred to light absorption unit (a.u.), and the horizontal axis of FIG. 9 is referred to absorption wavelength (nm). In FIG. 9, the BBMDC has the featured absorption peak around λ=332 nm, and the P3HT has the featured absorption peak around λ=522 nm.

According to the result of FIG. 9, as the proportion of the BBMDC blended in the photoactive layer is increased, the intensity of the BBMDC featured absorption peak is increased, and the P3HT featured absorption peak has significant blue-shift phenomenon. In this example, as the proportion of the BBMDC blended in the photoactive layer is increased as P3HT and BBMDC having a weight ratio from 1/0.6 to 1/1.5, the P3HT featured absorption peak may be shifted from 522 nm to 487 nm, and the shoulder peak beside the P3HT featured absorption peak becomes unobvious. The reason of the blue-shift phenomenon in the UV-vis absorption spectrum is that the π-π* energy gap is increased due to inhibition of the P3HT crystallization by adding BBMDC and then destruction of P3HT molecules stacked in the coplanar configuration.

Reference is made to FIG. 10, which is an UV-vis absorption spectrum of the photoactive layer according to an embodiment of the present invention, and the photoactive layer is obtained by blending P3HT and BBMDC of EXAMPLE 2 in a weight ratio of 1:0.6, 1:0.8, 1:1, 1:1.2, or 1:1.5 with annealing treatment. The vertical axis of FIG. 10 is referred to light absorption unit (a.u.), and the horizontal axis of FIG. 10 is referred to absorption wavelength (nm). In FIG. 10, the BBMDC has the featured absorption peak around λ=332 nm, and the P3HT has the featured absorption peak around λ=522 nm.

According to the result of FIG. 10, the intensity of the BBMDC featured absorption peak is changed less after annealing treatment. In comparison with FIG. 9 that is not subjected to annealing treatment, the annealing result of FIG. 10 shows that the P3HT featured absorption peak has little red-shift phenomenon but the corresponding intensity was significantly increased with increasing the proportion of the BBMDC blended in the photoactive layer. For example, the weigh ratio of P3HT/BBMDC changed from 1/0.6 to 1/1.5, the P3HT featured absorption peak shifted from 487 nm to 505 nm, resulting in red-shift phenomenon. In addition, the shoulder peak beside the P3HT featured absorption peak in FIG. 10 becomes more obvious than the one in FIG. 9. The reason of the red-shift phenomenon is that P3HT molecules stacked more compactly in the coplanar configuration, resulting in the stronger interaction between the P3HT molecules, so as to make carriers to jump and transfer easily between molecules. Besides, decreased π-π* energy gap also causes non-localized electrons of the molecular chain to transfer easily between π-π* states, resulting in red-shift occurrence in FIG. 10. The annealing treatment is responsible for the crystal intensity of P3HT. In the words, the crystallization of P3HT was enhanced after annealing, resulting in the regularly arrangement of P3HT molecules.

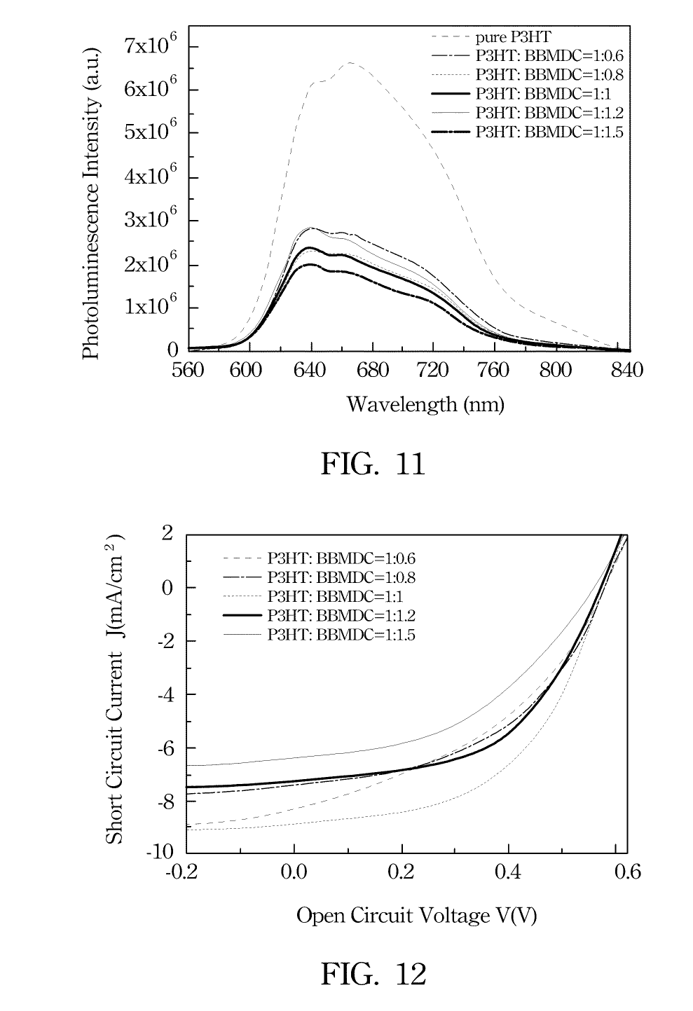

Reference is made to FIG. 11, which is a photoluminescence spectrum of the photoactive layer according to the present invention, and the annealing photoactive layer is composed of various weight ratio (1:0.6, 1:0.8, 1:1, 1:1.2, or 1:1.5, respectively) of P3HT/BBMDC in EXAMPLE 2. The vertical axis of FIG. 11 is referred to photoluminescence intensity (a.u.), and the horizontal axis of FIG. 11 is referred to absorption wavelength (nm). According to the result of FIG. 11, as the proportion of the BBMDC blended in the photoactive layer is increased, the photoluminescence intensity of the P3HT polymer is decreased for approximately 75%. This is explained by the fast charge transformations from the main chain of the P3HT polymer to BBMDC, due to the better electron-accepting property of BBMDC material, resulting in the less exciton recombination and the photoluminescence of P3HT itself.

Reference is made to FIG. 12, which is a current-voltage curve of the organic thin-film solar cell according to an embodiment of the present invention, the photoactive layer is obtained by blending P3HT and BBMDC of EXAMPLE 2 in various weight ratio (1:0.6, 1:0.8, 1:1, 1:1.2, or 1:1.5, respectively) with annealing treatment, and the current-voltage data are measured under irradiation of 100 mW/cm² light source with AM1.5G filter. The vertical axis of FIG. 12 is referred to short circuit current (Isc; mA/cm²), and the horizontal axis of FIG. 12 is referred to open circuit voltage (Voc; V). According to the result of FIG. 12, the open circuit voltage (Voc) was almost the same, even though the organic thin-film solar cells were prepared by various weight ratios of P3HT/BBMDC. However, the organic thin-film solar cell prepared by 1:1 weight ratio of P3HT/BBMDC has larger short circuit current (Isc).

Moreover, reference is made to Table 1, which is an optoelectrically performance data of the organic thin-film solar cell according to an embodiment of the present invention. According to the result of Table 1, the organic thin-film solar cell prepared by the weight ratio of 1:1 in P3HT/BBMDC blending has better performance on short circuit current (Isc), open circuit voltage (Voc), fill factor (FF) and solar energy to electricity conversion efficiency (n), which are 0.58 V, 8.9 mA/cm², 51.0% and 2.6%, respectively.

TABLE 1

| Material of Photo- | Weight | | I_{sc} | | |
|---|---|---|---|---|---|
| active Layer | ratio | V_{oc} (V) | (mA/cm²) | FF (%) | η (%) |
| P3HT/BBMDC | 1:0.6 | 0.59 | 8.33 | 39.6 | 1.9 |
| P3HT/BBMDC | 1:0.8 | 0.58 | 7.45 | 47.3 | 2.1 |
| P3HT/BBMDC | 1:1 | 0.58 | 8.90 | 51.0 | 2.6 |
| P3HT/BBMDC | 1:1.2 | 0.57 | 7.30 | 51.9 | 2.2 |
| P3HT/BBMDC | 1:1.5 | 0.56 | 6.45 | 48.3 | 1.8 |

Reference is made to Table 2, which is an optoelectrically performance data of the organic thin-film solar cell according to another embodiment of the present invention, the fullerene derivative utilized in the photoactive layer may include straight-chained C₂, C₄, C₈, or C₁₀ alkyl group, respectively, and the photoactive layer is obtained by blending P3HT and the aforementioned fullerene derivatives in 1:1 weight ratio with annealing. According to the result of Table 2, the fullerene derivatives having straight-chained C₂₋₁₀ alkyl groups can be applied to produce the photoactive layer. In another embodiment, the organic thin-film solar cell produced by the fullerene derivatives having straight-chained C₄₋₁₀ alkyl groups has solar energy to electricity conversion efficiency (η) higher than the one produced by the fullerene derivatives having straight-chained C₂ alkyl group.

TABLE 2

| Carbon number of | | | | |
|---|---|---|---|---|
| Straight-chained Alkyl Group | | I_{sc} | | |
| of Fullerene Derivative | V_{oc} (V) | (mA/cm²) | FF (%) | η (%) |
| C₂ | 0.53 | 1.90 | 46.3 | 0.47 |
| C₄ | 0.55 | 8.13 | 56.2 | 2.51 |
| C₈ | 0.56 | 7.68 | 57.8 | 2.49 |
| C₁₀ | 0.58 | 8.90 | 51.0 | 2.6 |

Reference is made to Table 3, which is an optoelectrically performance data of the organic thin-film solar cell according to a further embodiment of the present invention, and the photoactive layer is obtained by blending P3HT and the aforementioned fullerene derivatives in 1:1 weight ratio with annealing under different annealing temperature for approximately 10 minutes. According to the result of Table 3, the organic thin-film solar cell may be produced by using the electrode subjected to annealing treatment under 20° C. to 200° C. for 10 minutes. In another embodiment, the organic thin-film solar cell may be also produced by using the electrode subjected to annealing treatment under 100° C. to 150° C. for 10 minutes.

TABLE 3

| Annealing | | | | |
|---|---|---|---|---|
| Temperature | V_{oc} (V) | I_{sc} (mA/cm²) | FF (%) | η (%) |
| Room | 0.20 | 5.32 | 30.9 | 0.33 |
| Temperature | | | | |
| 100° C. | 0.51 | 7.17 | 50.7 | 1.85 |
| 150° C. | 0.55 | 8.13 | 56.2 | 2.51 |
| 200° C. | 0.48 | 1.25 | 17.7 | 0.11 |

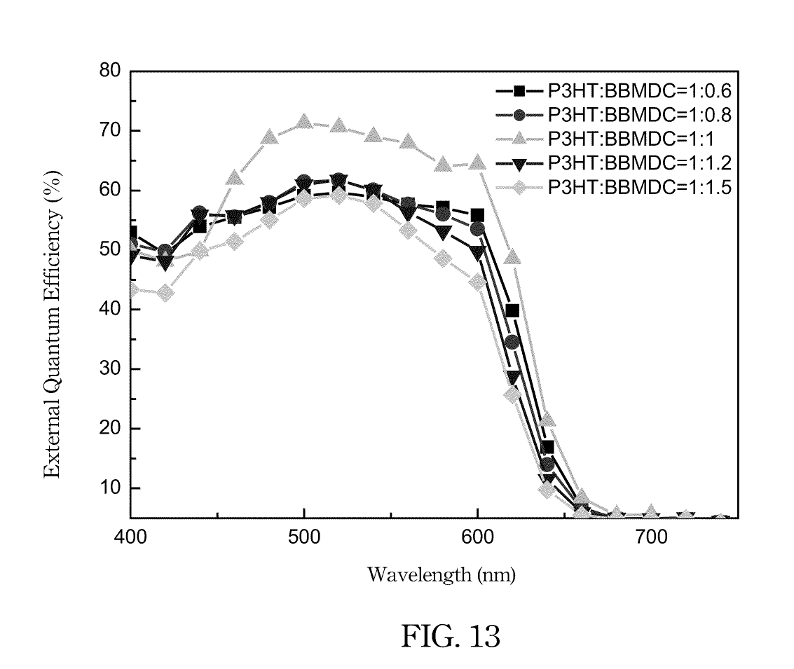

Reference is made to FIG. 13, which is an external quantum efficiency (EQE) spectrum of the organic thin-film solar cell according to an embodiment of the present invention, and the photoactive layer of the organic thin-film solar cell is obtained by blending P3HT and BBMDC of EXAMPLE 2 in various weight ratios (1:1.5, 1:1.2, 1:1, 1:0.8, or 1:0.6, respectively) with annealing. The vertical axis of FIG. 13 is referred to EQE (%), and the horizontal axis of FIG. 13 is referred to wavelength (nm). According to the result of FIG. 13, the EQE value of the organic thin-film solar cell produced by 1:1 weight ratio of P3HT/BBMDC is at least 5-10% higher than the one produced by other weight ratios of P3HT/BBMDC between 400 nm and 600 nm, and the EQE value of the organic thin-film solar cell produced by 1:1 weight ratio of P3HT/BBMDC is approximately 70% higher than the one produced by other weight ratios of P3HT/BBMDC at 520 nm. The similar conclusion can be drawn from the EQE spectrum of FIG. 13 and the UV-vis absorption spectrum of FIG. 9. Additionally, the organic thin-film solar cell produced by 1:1 weight ratio of P3HT/BBMDC also has more current density evidenced by FIG. 13.

In brief, the fullerene derivative for electron acceptor of the present invention introduces a benzylalkyl group therein for increasing its affinity of the fullerene derivative with electron donors, and it introduces the alkyl group for increasing its solubility in an organic solvent. Thereby, the fullerene derivative is facilitated to enhance the solar energy to electricity conversion efficiency of the resultant organic thin-film solar cell.

By the way, it is necessarily supplemented that, the fullerene derivative having specific structures, specific electron donor, specific hole-transferring material, the specific transparent electrode and the like are employed as exemplary embodiments in the present invention for evaluating the organic thin-film solar cell of the present invention, however, as is understood by a person skilled in the art, the fullerene derivative having different structures, different electron donors, different hole-transferring material and different transparent electrode can be employed in the present invention and be any combined thereof rather than limiting to the aforementioned examples.

According to the preferred embodiments of the present invention, the aforementioned fullerene derivative for electron acceptor and the organic thin-film solar cell manufactured by using the same in the present invention utilize the fullerene derivative serving as n-type organic polymer in the photoactive layer of the organic thin-film solar cell. The fullerene derivative introduces benzylalkyl and alkyl groups for increasing its affinity with electron donors, and it introduces an alkyl group for increasing its solubility in an organic solvent. Additionally, an annealing process can be employed to improve the crystallization and to reduce the phase separation state of the fullerene derivative and the electron acceptor. Thereby, the fullerene derivative is facilitated to enhance the solar energy to electricity conversion efficiency of the resultant organic thin-film solar cell.

As is understood by a person skilled in the art, the foregoing preferred embodiments of the present invention are illustrated of the present invention rather than limiting of the present invention. It is intended to cover various modifications and similar arrangements included within the spirit and scope of the appended claims. Therefore, the scope of which should be accorded to the broadest interpretation so as to encompass all such modifications and similar structure.

1. A fullerene derivative for electron acceptor represented by a chemical formula (I) as follows: 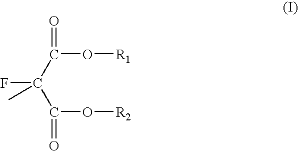
wherein F is fullerene, R₁ is independently selected from the group consisting of straight, branched or cyclic-chained C₂₋₁₀ alkyl groups, R₂ is a group of C₆H₅—CₙH₂ₙ—, and n of the R₂ is 1 to 3.

2. The fullerene derivative for the electron acceptor according to claim 1, wherein the F is C₆₀₋₈₄ fullerene.

3. The fullerene derivative for the electron acceptor according to claim 1, wherein the F is C₆₀ fullerene.

4. The fullerene derivative for the electron acceptor according to claim 1, wherein the R₁ is independently selected from the group consisting of straight-chained C₄₋₁₀ alkyl groups.

5. The fullerene derivative for the electron acceptor according to claim 1, wherein the R₁ is a butyl group.

6. The fullerene derivative for the electron acceptor according to claim 1, wherein the n of the R₂ is 1.

7. A fullerene derivative for electron acceptor represented by a chemical formula (I) as follows: 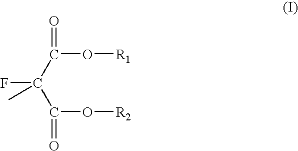
wherein F is C₆₀ fullerene, R₁ is a butyl group, and R₂ is a benzyl group.

8. A method of manufacturing fullerene derivative for electron acceptor, comprising:
reacting 2,2-dimethyl-1,3-dioxane-4,6-dione represented by a chemical formula (IV) with a first alcohol, so as to obtain a first intermediate product, wherein the first intermediate product is represented by a chemical formula (II): 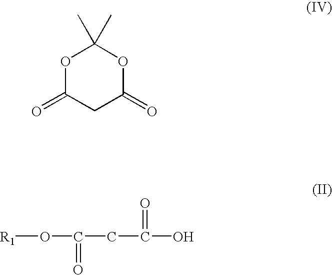
wherein R₁ is independently selected from the group consisting of straight-chained C₂₋₁₀ alkyl groups;
esterifying a second alcohol with the first intermediate product, so as to obtain a second intermediate product, wherein the second intermediate product is a malonic ester derivative, and the second intermediate product is represented by a chemical formula (III) as follows: 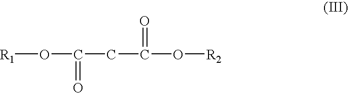
wherein R₂ is a group of C₆H₅—CₙH₂ₙ—, and n of the R₂ is 1 to 3; and
subjecting the second intermediate product and a fullerene to perform a Bingel reaction, so as to obtain the fullerene derivative, wherein the fullerene derivative is represented by chemical formula (I) as follows: 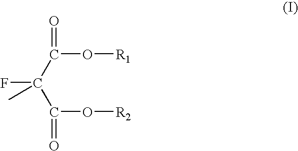
wherein F is fullerene.

9. The method of manufacturing the fullerene derivative for the electron acceptor according to claim 8, wherein the F is C₆₀₋₈₄ fullerene.

10. The method of manufacturing the fullerene derivative for the electron acceptor according to claim 8, wherein the F is C₆₀ fullerene.

11. The method of manufacturing the fullerene derivative for the electron acceptor according to claim 8, wherein the R₁ is independently selected from the group consisting of straight-chained C₄₋₁₀ alkyl groups.

12. The method of manufacturing the fullerene derivative for the electron acceptor according to claim 8, wherein the R₁ is a butyl group.

13. The method of manufacturing the fullerene derivative for the electron acceptor according to claim 8, wherein the n of the R₂ is 1.

14. An organic thin-film solar cell, comprising:
a light-transmitting electrode;
a hole-transferring layer disposed on the light-transmitting electrode;
a photoactive layer disposed on the hole-transferring layer, wherein the photoactive layer comprises electron acceptor and electron donor, and the electron acceptor is represented by a chemical formula (I) as follows: 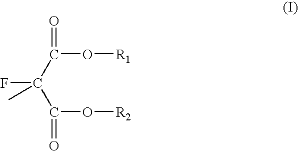
wherein F is fullerene, R₁ is independently selected from the group consisting of straight, branched or cyclic-chained C₂₋₁₀ alkyl groups, R₂ is a group of C₆H₅—CₙH₂ₙ—, and n of the R₂ is 1 to 3; and
a metal electrode disposed on the photoactive layer.

15. The organic thin-film solar cell according to claim 14, wherein the F is C₆₀₋₈₄ fullerene.

16. The organic thin-film solar cell according to claim 14, wherein the F is C₆₀ fullerene.

17. The organic thin-film solar cell according to claim 14, wherein the R₁ is independently selected from the group consisting of straight-chained C₄₋₁₀ alkyl groups.

18. The organic thin-film solar cell according to claim 14, wherein the R₁ is a butyl group.

19. The organic thin-film solar cell according to claim 14, wherein the n of the R₂ is 1.

20. The organic thin-film solar cell according to claim 14, wherein the electron donor is a conjugated polymer, and the conjugated polymer is poly(3-hexylthiophene) (P3HT).

21. The organic thin-film solar cell according to claim 20, wherein a weight ratio of the electron donor to the fullerene derivative is 1:0.2 to 1:5.

22. The organic thin-film solar cell according to claim 20, wherein a weight ratio of the electron donor to the fullerene derivative is 1:1.

23. The organic thin-film solar cell according to claim 14, wherein the hole-transferring layer comprises poly(3,4-ethylenedioxy-thiophene) (PEDOT): poly(styrene sulfonate) (PSS).

24. The organic thin-film solar cell according to claim 14, wherein the light-transmitting electrode is a patterned circuit.

25. The organic thin-film solar cell according to claim 14, wherein the metal electrode is made of aluminum or calcium.

26. A method of manufacturing organic thin-film solar cell, comprising:
forming a photoactive layer on a light-transmitting electrode, where the photoactive layer comprises electron donor and electron acceptor, and the electron acceptor is represented by a chemical formula (I) as follows: 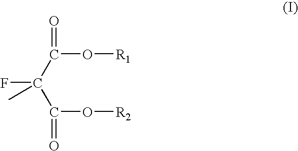
wherein F is fullerene, R₁ is independently selected from the group consisting of straight, branched or cyclic-chained C₂₋₁₀ alkyl groups, R₂ is a group of C₆H₅—CₙH₂ₙ—, and n of the R₂ is 1 to 3; and
forming a metal electrode on the photoactive layer.

27. The method of manufacturing the organic thin-film solar cell according to claim 26, wherein the F is C₆₀₋₈₄ fullerene.

28. The method of manufacturing the organic thin-film solar cell according to claim 26, wherein the F is C₆₀ fullerene

29. The method of manufacturing the organic thin-film solar cell according to claim 26, wherein the R₁ is independently selected from the group consisting of straight-chained C₄₋₁₀ alkyl groups.

30. The method of manufacturing the organic thin-film solar cell according to claim 26, wherein the R₁ is a butyl group.

31. The method of manufacturing the organic thin-film solar cell according to claim 26, wherein the n of the R₂ is 1.

32. The method of manufacturing the organic thin-film solar cell according to claim 26, wherein the electron donor is a conjugated polymer, and the conjugated polymer is P3HT.

33. The method of manufacturing the organic thin-film solar cell according to claim 26, wherein a weight ratio of the electron donor to the fullerene derivative is 1:0.2 to 1:5.

34. The method of manufacturing the organic thin-film solar cell according to claim 26, further comprising a hole-transferring layer formed on the light-transmitting electrode, and the hole-transferring layer comprises PEDOT: PSS.

35. The method of manufacturing the organic thin-film solar cell according to claim 26, wherein the light-transmitting electrode is a patterned circuit.

36. The method of manufacturing the organic thin-film solar cell according to claim 26, further performing an annealing step after the photoactive layer is formed.

37. The method of manufacturing the organic thin-film solar cell according to claim 36, wherein the annealing step is performed under 20° C. to 250° C. for 1 minute to 60 minutes.

38. The method of manufacturing the organic thin-film solar cell according to claim 36, wherein the annealing step is performed under 80° C. to 170° C. for 5 minutes to 20 minutes.

39. The method of manufacturing the organic thin-film solar cell according to claim 26, wherein the metal electrode is made of aluminum or calcium.

40. The method of manufacturing the organic thin-film solar cell according to claim 26, wherein the step of forming the metal electrode is to evaporate the metal on the photoactive layer.
